(19) United States
(12) Patent Application Publication
(10) Pub. No.: US 2005/0260173 A1
(43) Pub. Date: Nov. 24, 2005

(54) Arrrangement for using bioactive or osteoinductive material to build up a bone-based lateral support for implants in the jaw bone

(75) Inventor: Jan Hall, Goteborg (SE)

Correspondence Address: CONNOLLY BOVE LODGE & HUTZ LLP, SUITE 800, 1990 M STREET NW, WASHINGTON, DC, 20036-3425, US

(73) Assignee: Nobel Biocare AB (publ.), Goteborg (SE)

(21) Appl. No.: 10/521,153

(22) PCT Filed: Jun. 26, 2003
(86) PCT No.: PCT/SE03/01107

§ 371 Date: Jul. 14, 2005

(30) Foreign Application Priority Data
Jul. 25, 2002 (SE) 0202316-6

(51) Int. Cl. A61K 45/00; A61F 2/02

(52) U.S. Cl. 424/93.7; 623/17.17

(57) ABSTRACT

With the aid of bioactive or osteoinductive substances (10), a bone-based lateral support is built up for an implant (6) arranged in an assigned jaw bone hole in jaw bone (2) which extends defectively or irregularly. The implant is arranged so that it can be completely or partially covered by soft tissue, and possibly periosteum, or by a unit applied to the jaw bone and/or the implant. One or more spaces can in this way be formed on the lateral surface of the implant. Body fluid penetrates from or via the jaw bone and optionally the periosteum into the respective space. The osteoinductive or bioactive substances consist of matrix molecules, growth factors and differentiation factors and/or peptides with growth-stimulating properties, all called GSS, arranged in or the implant. Body fluid with cells is secreted and said GSS is released during an incorporation stage and forms, by interaction, new bone for said bone-based lateral support. A greater freedom of choice is thus made possible for the implant in defective or irregularly extending jaw bones, without compromising the stability or esthetic appearance of the implant.

The present invention relates to an arrangement for using bioactive or osteoinductive material to build up a bone-based lateral support for one or more implants arranged in assigned jaw bone holes. The invention is preferably used in conjunction with defectively or irregularly extending jaw bone, where the soft tissue of the jaw bone, sometimes combined with a separate unit, for example a polymeric and preferably stiff membrane, can be completely or partially drawn over the implant. In a completely or partially covering position for the implant or implants, the latter form one or more spaces together with the upper or side surfaces of the jaw bone and the soft tissue with or without periosteum (its underside) and/or the unit, and body fluids pass in from or via the periosteum and said jaw bone surface or jaw bone surfaces to said space or spaces.

The use of implants in jaw bone holes for supporting various dental fixtures is already known. Viewed in the horizontal plane of the jaw bone, the hole/implant is normally placed near the center line. At defects or irregularities in the jaw bone, the implant has to be offset either in the lateral direction or along the arc of the jaw bone so that the implant is given a position where, in its assigned jaw bone hole, it is surrounded by stable bone or stable bone formation. It is also known to insert two or more implants along the arc of the jaw bone and to use the implants as supports for a bridge construction or the like. In connection with the known implant, it is also already known to generally use bone substitute for the purpose of building bone mass around the implant when it has been screwed into the jaw bone hole. Examples of bone substitute which may be mentioned are autologous bone, allogenic bone, xenografts and/or synthetic preparations.

In the patent applications SE 9901972-1 and SE 9901973-9 previously submitted by the same Applicant and by the same inventor as in the present application, it is proposed that osteoinductive material be applied to the implant, for example on an outer surface with an outer porous oxide layer, or an outer thread which can be provided with porous oxide layer, and the implant can be self-tapping or is screwed into a threaded hole. The bioactive or osteoinductive materials can be applied in one or more layers and released material can cooperate with body fluid which occurs in the layer or the narrow gap between the jaw bone and the implant. Reference may also be made to the article published by, inter alia, the inventor of the present patent application and entitled “Properties of a New Porous Oxide Surface on Titanium Implants, Volume 1: The Oxidized Titanium Surface, Applied Osseointegration Research”. It is also known to adapt the diameter of the jaw bone hole to the diameter of the implant as a function of the quality of the jaw bone. It is also known to use, in connection with the implant, angled spacers which are intended to compensate for positional changes and inclinations of the implant. The implant can consist of titanium or another biocompatible material.

There is a need to be able to create a bone structure which allows the implant to be placed more ideally in conjunction with the arc of the jaw bone and so that the jaw bone, for example at said defects or irregularities, can permit implant applications where these are not initially surrounded by the hole wall or have a relatively great degree of exposure. There is a need to be able to adapt the implant positions to certain surfaces or outer thread parts which are more exposed in the circumferential and/or longitudinal direction(s) than other surfaces or outer thread parts in an initial stage. Despite the ideal application, the stability of the implant ought to be comparable to the case where the implant is laterally offset or longitudinally offset to a position where it is completely surrounded by the wall of the hole in the jaw bone.

The object of the present invention is to solve these problems among others and to realize implant structures which permit, from the point of view of their appearance, a considerable improvement compared to the case where the implant is offset laterally and/or longitudinally.

There is also a need to prevent the situation where the space formed by the soft tissue and possible periosteum, jaw bone and implant collapses and is filled with soft tissue, for example on account of stresses during the incorporation process. In some cases it may be important to avoid excessively large doses of osteoinductive material in connection with narrow gaps between the implant and the wall of the hole in the jaw bone. Such high doses may, in an initial stage, have an effect which counteracts the process of new bone formation. It is also important to be able to stimulate new bone formation with the aid of the geometry of the spaces used for growth. The space in the jaw bone and the unit must therefore be able to be chosen with a geometry which permits effective new bone formation. The invention solves these problems too.

There is a need for surgeons and other treating personnel to have a greater freedom of choice in positioning the implants more independently of the jaw bone status than previously, but without the stability of the incorporated implant being compromised. The invention solves this problem too.

The feature which can principally be regarded as characterizing an arrangement according to the invention is that the bioactive or osteoinductive material consists of growth-stimulating substance or substances (here called GSS), arranged in or on the implant, preferably on one or more outer side surfaces or one or more outer thread parts which in an initial stage are exposed from the jaw bone. Said GSS, in a stage of incorporation following the initial stage, passes into each closed space and interacts with the aforementioned cells, for example the stem cells, thus forming the bone-based lateral support for the implant. Different types of GSS can be used, and examples of GSS which may be mentioned are matrix proteins, growth factors and differentiation factors and/or peptides with growth-stimulating properties.

In one embodiment, the invention is used for an implant with a position for the jaw bone's imagined horizontal plane which is offset in relation to the center line of the jaw bone in the horizontal plane so that the implant in said initial stage has first side surface parts or outer thread parts having a greater degree of exposure than other side surface parts or outer thread parts. The bone-based new formation is intended, in the stage of incorporation, to give the first side surface parts or outer thread parts an increased degree of bone coverage or increased degrees of bone coverage. In one embodiment, two or more implants can be arranged along the horizontal extent of the jaw bone in assigned jaw bone holes. Said implants are in this case arranged at defects or irregularities in depth and/or the lateral direction or lateral directions. In the stage of incorporation, the jaw bone's defects or irregularities are substantially filled and the implant is given substantially the same degree of coverage with bone all round it after the stage of incorporation has been completed. In the case of a jaw bone strongly degenerated in the vertical direction, all the implants in one embodiment can be given bone-based lateral supports extending substantially identically in the height direction.

In one embodiment, first portions of each implant have a greater degree of exposure than other portions of the implant or implants. Said first portions are in this case coated with more GSS than the other portions. The aforementioned unit made of, for example, stiff and/or polymeric membrane can be used temporarily or can be included permanently in the fixed installation. The unit can be attached to the jaw bone and/or the implant, for example by screw(s), during at least the initial stage and the stage of incorporation. The unit can have an internally curved surface which, when the unit is in the applied position, is directed toward the side surface or outer thread of the respective implant. The unit can be designed with an upper part which extends completely or partially over the upper or outer surface of the implant. The respective outer surface or outer thread exposed in the initial stage extends between 20-180°, preferably 30-120°, viewed in the circumferential direction of the implant. Said outer surface exposed in the initial stage can also extend 20-80%, preferably 30-70%, along the height direction of the implant.

Further embodiments are set out in the attached dependent claims.

By means of what has been proposed above, it is possible to achieve optimum implant positions, especially from the point of view of appearance, in defective or irregular jaw bones, without the stability of the implant being compromised. Known and well established materials can be used for the implant, for example titanium, ceramic, etc. The invention functions for one or more implants, and in the case of several implants these can be arranged one after another along the horizontal extent of the defective or irregular jaw bone. The invention functions for portions exposed to greater or lesser extents in the initial stage. A separate unit or the stiff and/or polymeric membrane can be used to secure the actual space in which the new formation of dentine takes place by means of GSS. The unit/the membrane can be used temporarily or as a continuous/permanent fixture. The unit/membrane can be secured by means of screws, with arms or structured parts, etc., and made of titanium, plastic, etc.

A presently proposed embodiment of an arrangement having the features characteristic of the invention will be described below with reference to the attached drawings, where

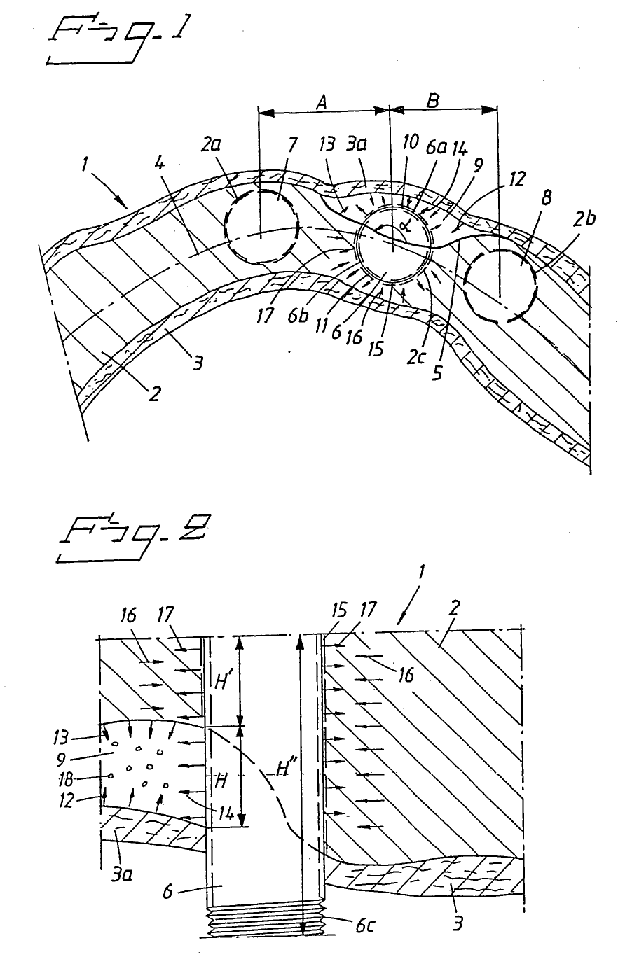

FIG. 1 shows, in horizontal section, a lower jaw bone with a defect or irregularity, in or on which an implant is to be anchored, said figure also showing positions for the implant which have been indicated in the prior art,

FIG. 2 shows, in vertical section, an implant fitted in a jaw bone hole (in the upper jaw) and where a space for a lateral support formed by new bone is included in the implant's anchoring,

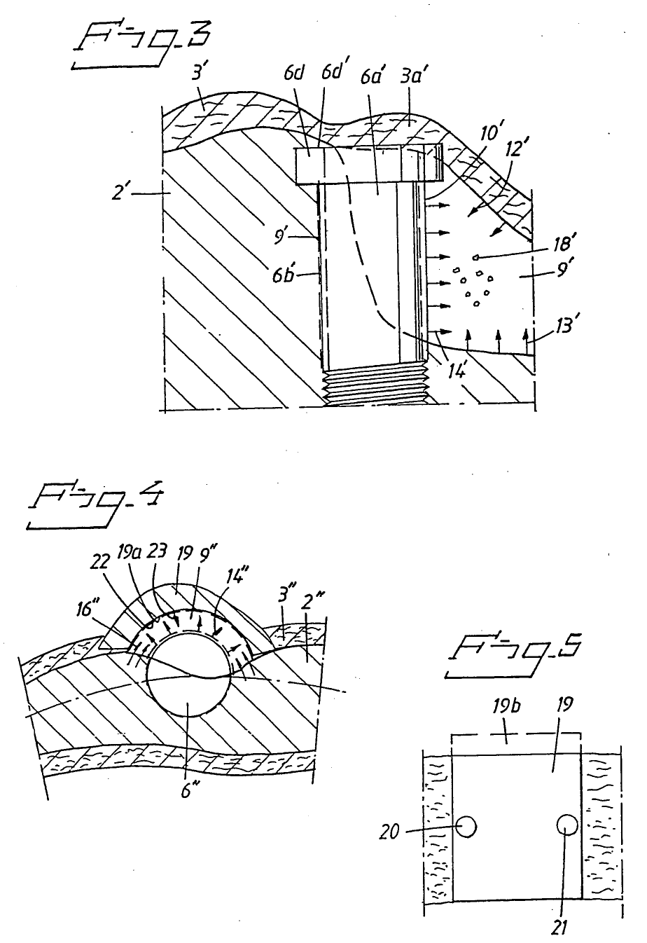

FIG. 3 shows, in vertical section, an implant fitted in a lower jaw, said lower jaw having a defect or irregularity different than the defect or irregularity in FIG. 2,

FIG. 4 shows, in horizontal section, the use of a unit applied to the dentine, for example a membrane made of titanium, or plastic, etc., and

FIG. 5 shows, in a side view, the extent of the unit.

In FIG. 1, a lower jaw bone is shown diagrammatically by 1. The lower jaw bone itself is indicated by 2, and the soft tissue of the jaw bone, with underlying periosteum, is shown by 3. Generally speaking, periosteum may be completely or partially absent, but in the present case it is assumed to be present, although not specifically pointed out. The arc-shaped extent of the jaw bone in the horizontal direction is shown by 4. The jaw bone is provided with a defect or an irregularity which is indicated by 5. When fitting implants optimally in a jaw bone hole in the jaw bone, it may be necessary from the point of view of appearance, the point of view of installation, etc., to place the implant at the irregularity or defect 5. In the previously known technique, this freedom of positioning has not been possible and it has often been necessary to fit the implant in a position which is offset in relation to the defect or irregularity and where more bone mass for the implant and the jaw bone hole has been available. Alternatively, it has been necessary to fill the space around the implant with bone substitute of various types. In FIG. 1, an implant 6 is optimally fitted at the defect or irregularity 5. Said previous laterally offset positions have been indicated by 7 and 8, and it will be seen that the implant position 7 has to be offset in relation to implant position 6 by a distance A for sufficient bone mass to be present at the partially shown jaw bone hole 2a and the implant 7 arranged therein. In an alternative offset to the position 8 and jaw bone hole 2b, the implant 6 has to be offset by a distance B. It will be appreciated that such offsets can affect the implant fixture from the point of view of appearance and that measures may be required in the actual dental fixture, which can include spacer sleeves, bridge construction, etc. When replacing a lost tooth in an otherwise intact row of teeth, it will also be appreciated that problems may arise when applying the implant for the lost tooth if a defect or irregularity is present in the jaw bone at the location of the lost tooth.

In accordance with the invention, the implant 6 is placed at the defect or irregularity 5 and the positions 7 and 8 are therefore not used. In accordance with the invention, a space 9 is created on the exposed side surface 6a. The angle for the exposed side surface is indicated by a in FIG. 1. In a preferred embodiment, the size of said angle can assume values of between 20 and 180°, preferably values in the range of 30-120°, viewed in the circumferential direction (i.e. in the plane in FIG. 1). The implant is provided with layers 10, 11 of GSS. In a preferred embodiment, the concentration of GSS in the layer on the exposed side surface or outer thread part 6a is greater than the layer 11 which is directed toward the jaw bone 2. It is thus possible to work with a predetermined angle position of the implant when it has been screwed or secured in position in the jaw bone/jaw bone hole 2c. For reasons of clarity, the concentration of GSS in said layers 10, 11 is symbolized by an unproportional thickness in FIG. 1. In the illustrative embodiment according to FIG. 1, a soft tissue and periosteum part 3a is drawn across the surface Ga exposed in relation to the jaw bone 2. Body fluid accumulates in a manner known per se in the space 9, this body fluid being secreted from or via body tissue, the jaw bone 2 and the soft tissue and periosteum 3, 3a. In a likewise known manner, this body fluid contains cells, and here reference may be made to the fact that the periosteum in particular supplies a large amount of stem cells. Said body fluid is symbolized in FIG. 1 by arrows 12 and 13. Said body fluid releases said GSS from the surface 6a of the implant and, through said secretion and release, a process or interaction is initiated for new formation of bone in the space 9. Thus, during a stage of incorporation of the implant 6, a lateral support is formed in the space 9, this lateral support consisting of newly formed bone, giving the lateral support a character corresponding to the compact bone mass, cf. the positions 7 and 8 for the implant. The defect or irregularity 5 is filled by the new bone formation. The process of release of GSS is symbolized by arrows 14 in the figure. In the nonexposed portions 6b of the implant, a process of new formation of bone takes place in a corresponding manner in a gap 15 between the side surface 6b of the implant and the wall of the hole 2c. In this case, the body fluid formed from the jaw bone is indicated by 16, and the release of GSS on the surface 6b is indicated by arrows 17. The layer 11 must not obtain a dose resulting in excessive reaction of GSS on the dentine 2, as this may in some cases involve a degeneration process of the bone formation. The implant can be used in accordance with said patent applications from the same Applicant and inventor. Thus, the outer surface in question, for example a threaded outer surface, can be arranged with an oxide layer having pores in which GSS is stored. In one embodiment, GSS can be used in combination with material containing calcium phosphate. In one embodiment, bone substitute known per se and available on the market can be used in combination with said GSS. In this connection, reference may be made to autologous bone, allogenic bone, xenografts and/or synthetic materials or substances.

In FIG. 2, corresponding parts and arrows have been indicated with the same reference numbers. The height of the exposed part 6a has been indicated by H, and the inner parts of the implant are surrounded by jaw bone 2. The implant's parts surrounded by jaw bone have been indicated by H′. The value of H can be 20-80% of the total height of the implant, which is symbolized in FIG. 2 by H″. The preference is for values in the rage of 30-70%. The implant can be provided with a thread 6c in a manner well known per se.

FIG. 3 shows an implant 6a′ arranged in a lower jaw bone. Parts in FIG. 3 corresponding to FIGS. 1 and 2 have been indicated in FIG. 3 with the same reference numbers with addition of a prime marker. As can be seen from FIG. 3, the defect or irregularity has another course which exposes outer surfaces or outer thread parts of the implant, different from the case according to FIG. 2. In this case, the soft tissue together with possible periosteum 3a′ has also been drawn across the upper parts 6d of the implant 6a′. The release and secretion functions correspond to those described above.

In accordance with FIGS. 4 and 5, a temporary or permanent unit 19 can be used to create the space 9″. In some cases the unit can be secured in the jaw bone 2′ by means of screws 20 and 21 or other securing means. The unit can consist of a polymeric or metal-based, stiff membrane and, like the implant, can be made of titanium, and in one embodiment it has an arcuate or semicircular inner surface 19a. Said inner surface can be provided with said material GSS. A release function of GSS can take place in cooperation with said body fluid secretion 16″ according to the above. The function of secretion from the unit 19 is symbolized by 23 in FIG. 4. The unit 19 can be provided with an upper part 19b which can extend in across the implant, i.e. across the top faces of the implant. The upper part 19b can also be provided on its top face with layers of GSS. The arcuate shape 19a has advantages for the growth function which is especially advantageous in the case of convex surfaces corresponding to the surface 19a. The coating of GSS 22 can also be combined with bone substitute in accordance with what has been described above in relation to the space 9, 9′.

The arc line 4 constitutes the ideal arc line, while the actual center line extending crookedly in a jaw bone with defects or irregularities is not shown in detail. This center line is referred to as the actual center in the horizontal plane. The upper or outer surface of the implant is indicated by 6d′ in FIG. 3.

The invention is not limited to the embodiment shown above by way of example, and instead it can be modified within the scope of the attached patent claims and the inventive concept.

Reference may be made here to patent applications submitted to the Swedish patent office on the same day as the present patent application and by the same Applicant and inventor. Said applications have the following titles:
a) “Arrangement for using osteoinductive or bioactive material to induce bone and/or increase the stability of implants in the jaw bone, and an implant intended for this purpose”.
b) “Arrangement for implants bearing growth-stimulating substance or substances, and one such implant”.
c) “Arrangement of two or more implants provided with growth-stimulating substance(s)”.
d) “Arrangement for increasing the stress resistance of implants, and one such implant”.

1. An arrangement for using bioactive or osteoinductive material to build up a bone-based lateral support (18,18′) for at least one implant (6) arranged in an assigned jaw bone hole (2c) and where the implant is arranged so that it can be completely or partially covered by soft tissue, with or without the periosteum of the jaw bone, or by a unit applied to the jaw bone, and where the implant, when completely or partially covered, forms one or more spaces together with the soft tissue and the possible periosteum and/or the unit and the upper or lateral surface(s) of the jaw bone in question, and cell-containing body fluid penetrates into this space or these spaces from at least said jaw bone, characterized in that the bioactive or osteoinductive material comprises GSS comprising matrix molecules, growth factors and differentiation factors and/or peptides with growth-stimulating properties, arranged in or on the implant, which GSS, in a stage of incorporation following the initial stage, passes into each closed space and interacts or integrates with said cells and thus forms the bone-based lateral support for the implant.

2. The arrangement as claimed in patent claim 1, characterized in that the jaw bone hole (2c) and thus the implant (6) have a position which is offset in relation to the real center line of the jaw bone in the horizontal plane, so that the implant in said initial stage has first side surface parts or outer thread parts which have a greater degree of exposure than other side surface parts or outer thread parts, and after the stage of incorporation the bone-based lateral support is intended to give the first side surface parts or outer thread parts an increased degree of bone coverage or increased degrees of bone coverage.

3. The arrangement as claimed in patent claim 1, characterized in that two or more implants which are arranged along the horizontal extent of the jaw bone in assigned jaw bone holes are arranged in conjunction with defects or irregularities in depth and/or the lateral direction(s), and in that, in the stage of incorporation, they substantially fill the jaw bone defects and irregularities and give the implant substantially the same degree of recessing after the stage of incorporation.

4. The arrangement as claimed in patent claim 1, characterized in that, in the case of a jaw bone greatly degenerated in the vertical direction, all the implants are given bone-based lateral supports extending substantially identically in the vertical direction.

5. The arrangement as claimed in patent claim 1, characterized in that, first portions (6a) of each implant with a greater degree of exposure than other portions (6b) of the implant or implants are covered with GSS with a greater or lesser degree of concentration of GSS than the other portions.

6. The arrangement as claimed in claim 1, characterized in that the unit (19) can be temporarily or permanently attached to the jaw bone, the unit, when temporarily attached, being applied during the initial and incorporation stages.

7. The arrangement as claimed in patent claim 1, characterized in that the unit has an internally curved surface (19a) which, when the unit is applied, is directed toward the side surface (6a) or outer thread part of the respective implant (6).

8. The arrangement as claimed in patent claim 1, characterized in that the unit has an upper part (19b) which completely or partially extends over the implant's upper or other surface (6d′).

9. The arrangement as claimed in claim 1, characterized in that, at its surface covered by the wall of the jaw hole (2c), the implant works with body fluid accumulation in the layer or the gap (15) between the implant and the wall (2c).

10. The arrangement as claimed in claim 1, characterized in that the implant's outer surface (6a) exposed in the initial stage extends between 20-180°, viewed in the circumferential direction of the implant.

11. The arrangement as claimed in claim 1, characterized in that the implant's outer surface exposed in the initial stage extends 20-180%, viewed in the height direction (H).

12. The arrangement as claimed in patent claim 1, characterized in that the unit is coated with GSS on its outer surface (6a) exposed toward the implant in the initial stage.

13. The arrangement as claimed in claim 1, characterized in that the implant's outer surface (6a) exposed in the initial stage extends between 30-120°, viewed in the circumferential direction of the implant.

14. The arrangement as claimed in claim 1, characterized in that the implant's outer surface exposed in the initial stage extends 30-70%, viewed in the height direction (H).

15. The arrangement as claimed in patent claim 2, characterized in that two or more implants which are arranged along the horizontal extent of the jaw bone in assigned jaw bone holes are arranged in conjunction with defects or irregularities in depth and/or the lateral direction(s), and in that, in the stage of incorporation, they substantially fill the jaw bone defects and irregularities and give the implant substantially the same degree of recessing after the stage of incorporation.

16. The arrangement as claimed in patent claim 2, characterized in that, in the case of a jaw bone greatly degenerated in the vertical direction, all the implants are given bone-based lateral supports extending substantially identically in the vertical direction.

17. The arrangement as claimed in patent claim 3, characterized in that, in the case of a jaw bone greatly degenerated in the vertical direction, all the implants are given bone-based lateral supports extending substantially identically in the vertical direction.

18. The arrangement as claimed in patent claim 2, characterized in that first portions (6a) of each implant with a greater degree of exposure than other portions (6b) of the implant or implants are covered with GSS with a greater or lesser degree of concentration of GSS than the other portions.

19. The arrangement as claimed in patent claim 3, characterized in that first portions (6a) of each implant with a greater degree of exposure than other portions (6b) of the implant or implants are covered with GSS with a greater or lesser degree of concentration of GSS than the other portions.

20. The arrangement as claimed in patent claim 4, characterized in that first portions (6a) of each implant with a greater degree of exposure than other portions (6b) of the implant or implants are covered with GSS with a greater or lesser degree of concentration of GSS than the other portions.
